(19) United States
(12) Patent Application Publication
(10) Pub. No.: US 2006/0163382 A1
(43) Pub. Date: Jul. 27, 2006

(54) Spray booth method and apparatus

(75) Inventors: Paul Spivak, Avon Lake, OH (US); Nicholas J. Mastandrea, Newbury, OH (US); Scott R. Thomason, Macedonia, OH (US); Timothy Niedel, Euclid, OH (US)

Correspondence Address: BROUSE MCDOWELL;A Legal Professional Association, Suite #500, 388 South Main Street, Akron, OH, 44311-4407, US

(73) Assignee: GREAT LAKES ENGINEERING & DESIGN, INC.

(21) Appl. No.: 10/805,689

(22) Filed: Mar. 22, 2004

Related U.S. Application Data
(60) Provisional application No. 60/456,744, filed on Mar. 21, 2003.

(51) Int. Cl. B05B 15/06; B05B 1/14; A01G 25/06; A62C 2/08

(52) U.S. Cl. 239/200; 239/548; 239/551

(57) ABSTRACT

A spray booth includes a fluid pump that draws fluid from one or more reservoirs. The pump propels the fluid to a plurality of nozzles and combines pressurized air to atomize the fluid for dispersion within the spray booth. The plurality of nozzles is synchronously actuated to spray the fluid in a sequential manner.

This U.S. Utility patent application Ser. No. claims priority from U.S. Provisional patent application, Ser. No. 60/456,744 filed on 21 Mar. 2003.

### FIELD OF ART

The present invention relates generally to a method and apparatus comprising a fixed plurality of atomizing spray nozzles configured to distribute an atomized liquid mixture with force within a designated area. More particularly, a preferred embodiment of the present invention is adapted to spray a tanning mixture upon a human person.

### BACKGROUND OF THE INVENTION

It is known in the art to provide a device for spraying various fluids on the human body. One application for applying fluids is related to sunless tanning procedures, wherein an associated fluid is sprayed onto the body for use in darkening the skin simulating the sun tanning process. One aspect of the subject matter relates to a spray containing booth wherein an entire individual may step into the booth and subsequently wherein the sunless tanning fluid is sprayed onto the body of the individual.

In some prior inventions related to the present subject matter, nozzles are included in the spray booth where the nozzles are moveably connected with respect to the walls of the spray booth. Typically this type of connection involves some type of motorized actuator which maneuvers the nozzles about during the dispersing of the atomized fluid onto the individual standing in the booth. In particular, this type of spray booth with moving nozzles inheritently includes maintenance problems that develop over time as the spray booth is used. What is needed is a spray booth that effectively and simply covers the human body with atomized fluid spray and yet it does not include nozzles that move with respect to the booth structure. The subject invention includes a series of spray nozzles that are engaged in succession to optimize the flow of spray onto an individual standing in the spray booth.

### SUMMARY OF THE PRESENT INVENTION

One aspect of the subject invention includes a spraying device, including an upright structure having a nozzle support portion, at least first and second spray nozzles being operatively attached to the nozzle support portion, means for operatively supplying an associated fluid to the at least first and second spray nozzles, a spray nozzle controlling means for use in selectively independently actuating the at least first and second spray nozzles respectively, wherein when the spraying device is engaged the at least first spray nozzle is sequentially actuated with respect to the at least second spray nozzle.

Another aspect of the subject invention includes an enclosure for use in containing the associated fluid, wherein the nozzle support portion is a wall member.

Another aspect of the subject invention includes an enclosure for use in containing the associated fluid, wherein the enclosure is a modular booth having a plurality of spray containing walls, a bottom booth portion, and a top booth portion.

Another aspect of the subject invention includes an enclosure for use in containing the associated fluid, wherein the at least first and second spray nozzles are at least first and second banks of spray nozzles.

Another aspect of the subject invention includes an enclosure for use in containing the associated fluid, wherein the at least first bank of spray nozzles are fixedly operatively attached with respect to the wall member.

Another aspect of the subject invention includes an enclosure for use in containing the associated fluid, wherein the means for operatively supplying an associated fluid comprises pumping means for use in conveying the associated fluid to the at least first and second banks of spray nozzles and at least a first reservoir operatively connected to the pumping means. The fluid can also be supplied using a pressurized fluid container.

Another aspect of the subject invention includes an enclosure for use in containing the associated fluid, further comprising a shut-off valve operatively communicated between the at least a first reservoir and the at least first spray nozzle.

Another aspect of the subject invention includes an enclosure for use in containing the associated fluid, wherein the shut-off valve is a check valve.

Another aspect of the subject invention includes an enclosure for use in containing the associated fluid, further comprising conduit for use in channeling the associated fluid, the conduit being operatively connected to the pumping means and the at least first and second spray nozzles, wherein the pumping means is a gear pump or any mechanical pump that continuously cycles the associated fluid, and further comprising a bypass valve for use in selectively channeling the associated fluid, the bypass valve being operatively communicated between the pump and the fluid containing enclosure.

Another aspect of the subject invention includes an enclosure for use in containing the associated fluid, further comprising at least a second reservoir operatively connected to the pumping means.

Another aspect of the subject invention includes a method for dispensing an associated fluid, comprising the steps of providing at least a first and second spray nozzles, wherein the at least a first spray nozzle is selectively independently actuate-able with respect to the at least a second spray nozzle, providing means for use in pumping an associated fluid from at least a first reservoir, the pumping means being operatively communicated to the at least first and second spray nozzles, providing controlling means operatively communicated to actuate the at least first and second spray nozzles, engaging the controlling means, spraying an associated fluid through the at least a first spray nozzle, and sequentially spraying the associated fluid through the at least a second nozzle.

Another aspect of the subject invention includes a method for dispensing an associated fluid, wherein before the step of spraying an associated fluid through the at least a first spray nozzle, further comprising the step of atomizing the associated fluid.

Another aspect of the subject invention includes a method for dispensing fluid, comprising the steps of providing at least a first spray nozzle, providing pumping means for use in pumping associated fluid, the pumping means being operatively communicated to the at least a first spray nozzles, providing controlling means operatively communicated to actuate the at least a first spray nozzles, providing first and second fluid reservoirs operatively communicated to the pumping means, selecting one of the first and second reservoirs from which to draw associated fluid from, engaging the controlling means and spraying the associated fluid through the at least a first spray nozzle.

Another aspect of the subject invention includes a method for dispensing fluid, further comprising the steps of purging the associated fluid from the at least a first spray nozzle and spraying associated fluid from the remaining of the one of the first and second reservoirs.

The spray booth may include multiple nozzles that are independently controlled or actuate-able via a logic processing controller, which may include a microprocessor and support peripheral circuitry. The nozzles, whether in groups or rows, or individual, may be selectively turned on to begin the flow of fluid through the nozzle. A pump, which may be a gear pump, cycles fluid to and from a reservoir until a solenoid valve is operated to allow pressurized fluid to flow to the nozzle. Simultaneously, pressurized air is caused to flow to the nozzle for use in atomizing the fluid as is dispersed through the nozzle. The banks of nozzles may be synchronized to spray the atomized fluid in succession. The sequence may be predefined as automatically controlled by the spray nozzle controller.

The advantage of the present invention over prior art inventions that use a moving atomized spray head, such as U.S. Pat. No. 6,305,384 to Laughlin, is that the present invention does not have any moving parts or motorized or mechanized mechanisms required to practice the invention. Thus, there are no motorized or moving parts to break down, or to come out of alignment. The multiple nozzle 14 array taught by the present invention thus is mechanically superior to the prior art, and it is much more reliable and simple to create, build, or perform. The present invention may also be configured to more rapidly distribute the atomized fluid product upon a person P within the booth where all of the nozzles 14 are configured to fire simultaneously. In contrast, the prior art moving single nozzle taught by Laughlin, must slowly move about the entire person and, therefore, it would take more time to distribute the same amount of atomized fluid upon a person P. Thus, it is apparent that the present invention will allow much more rapid distribution of atomized product, and will thus allow more people to be serviced by the present invention 2 over the same time period contrast servicing each person at a higher rate that the prior art spray booths.

### BRIEF DESCRIPTION OF THE DRAWINGS

FIG. 1 is a side perspective view of an embodiment of the present invention.

FIG. 2 is a front perspective illustration of a spray apparatus front panel according to the present invention.

FIG. 3 is rear perspective view of the panel of FIG. 2.

FIG. 4 is a detail perspective rear view of the panel of FIG. 3.

FIG. 5 is a detail perspective rear view of the panel of FIG. 3.

FIG. 6 is a side perspective view of a spray booth apparatus according to the present invention.

FIG. 7 is a perspective view of the spray booth.

FIG. 8 is a schematic representation of a nozzle with conduits connected to the nozzle and including shut-off valves.

### DESCRIPTION OF THE PREFERRED EMBODIMENT

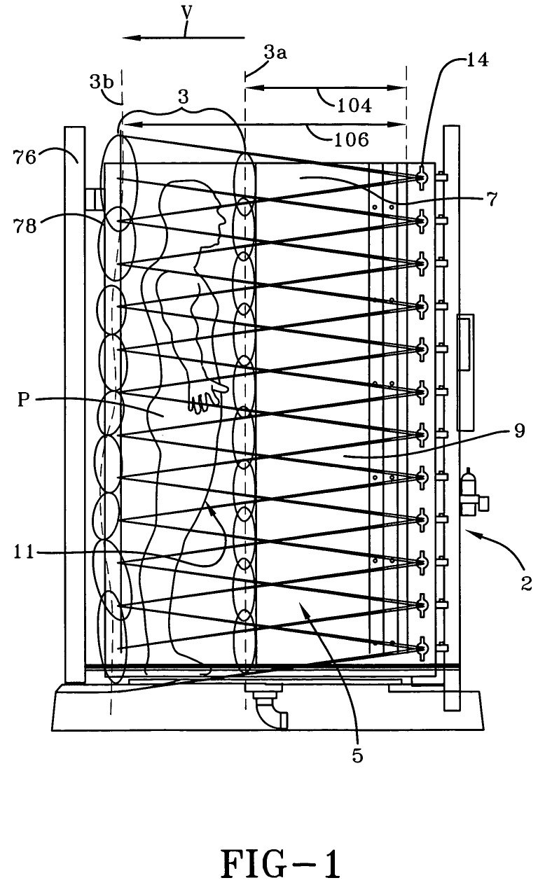

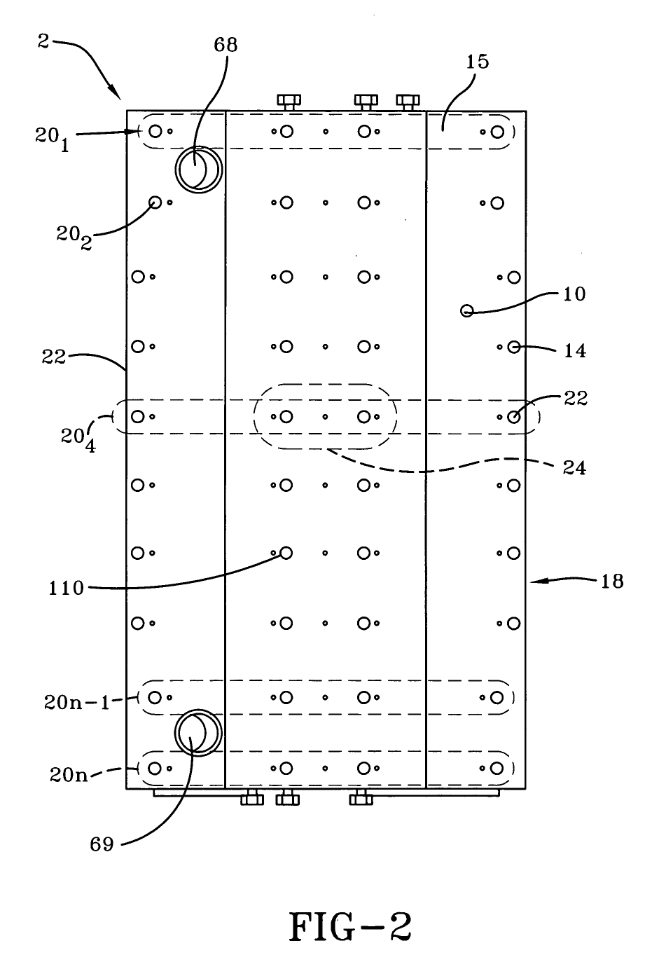

Referring now to the drawings wherein the showings are for purposes of illustrating a preferred embodiment of the invention only and not for purposes of limiting the same, FIG. 1 shows a side perspective view of an embodiment of the present invention. A person P is shown receiving a forceful atomized liquid and air mixture 5 within a preferred dispersion region 3 distributed by a spraying apparatus 2. The spraying apparatus 2 comprises a plurality of spray nozzles 14. As shown in FIG. 2, the nozzles 14 are preferably arranged in a matrix configuration 18 on a panel 15. In one embodiment, the matrix 18 is a four wide by ten high rectangular matrix pattern formed by forty nozzles 14, but it will be readily apparent that other configurations and number of nozzles 14 may be used.

With continued reference to FIGS. 1 and 2, the nozzles 14 forcefully project an atomized liquid mixture 5 toward the person P. An initial distribution area 7 of the atomized mixture, defined between the nozzles 14 and the preferred mixture region 3, comprises very turbulent and excessively forceful droplet and air current patterns moving at high velocities. As the atomized liquid and air mixture 5 is expelled from the nozzles 14 and travels through the initial area 7, it collides and mixes with the ambient air 9 present in the area. This forceful mixing reduces the volatility and high velocities present in the initial region 7 and more uniformly disperses and stabilizes the mixture 5, resulting in a wide and uniform mixture 5 within the engaging region 3. The engaging region is defined by the shape of the matrix 18. In the present embodiment, the front planar boundary 3a of the region 3 is parallel to and spaced a distance 104 from the matrix 18 of the nozzles 14. The rear boundary 3b of the region 3 is parallel to and spaced a distance 106 from the matrix 18. Thus, a person P standing within the region 3 and aligned generally parallel to the matrix 18 is struck upon the facing skin surfaces 11 with the resultant atomized mixture 5 with a reduced yet effective force. For one embodiment of the invention, each nozzle 14 compels an atomized fluid mixture at a rate of about 3.75 cubic feet per minute (cfm): for this rate, a preferred distance 104 is about twenty-seven inches, and distance 106 is about four feet. However, it is noted that any rate of spraying the atomized fluid mixture and any distance between the person P and the nozzles 14 may be chosen with sound engineering judgment.

With continued reference to FIGS. 1 and 2, a preferred embodiment of the present invention is adapted to spray a mixture 5 comprising a liquid tanning solution. While the present invention relates to a liquid tanning solution, it will be appreciated that the subject invention may disperse any type atomized fluid. Accordingly, it is desirable, and a significant advantage of the present invention, that the mixture 5 strike the person P within the engaging region 3 with some force, in order to facilitate penetration of the skin surface 11 of the person P by the mixture 5, and thereby help the skin 11 to absorb the tanning mixture 5. This type of distribution method and apparatus is preferred over one that may just convey a low velocity or foggy type mist onto a person P, since in the latter case the fluid will not penetrate into the skin as readily as with the force conveyed with the current invention. By utilizing a fixed matrix array of nozzles 14, the apparatus 2 can be configured to locate each nozzle 14 at a uniform desired distance from the subject person P, and thereby select the amount of force exerted by the atomized and expelled mixture 5 upon the skin surface 11. The present invention also eliminates the need for a fan or negative pressure element to draw an atomized fog or mist across the person P, since the atomized mixture 5 is compelled with force from the nozzles 14 into a person P aligned within the engagement region 3.

Another advantage of the present embodiment is that the nozzles 14 work cooperatively in shaping the atomized mixture compelled through the engagement region 3. As the atomized mixture 5 moves through the initial turbulent region 7, the ambient air 9 present within this area mixes with and stabilizes the propelled atomized mixture 5 by absorbing kinetic energy and slowing down the velocity of the liquid droplets therein. The neighboring atomized mixture 5 plumes propelled by the neighboring nozzles 14 help to compel the energy and motion of the propelled mixture along a common vector V toward the person P. Thus, no other structural elements are required to direct or otherwise contain the atomized mixture 5 through the engagement region 3. Where no ambient wind currents are present, the subject invention may thus “spray” the person P without the need for any structural closure or containment.

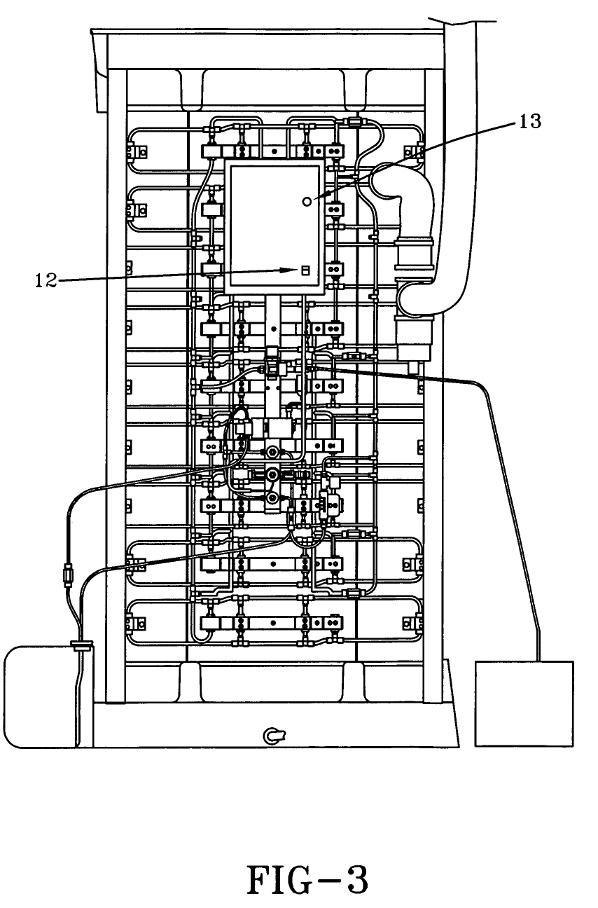

With continued reference to FIGS. 1 and 2, in a preferred embodiment of the invention, a person P is sprayed in a “four-pass” discreet spray sequence to ensure complete body coverage; one pass on the front side, a second pass on the left or right side, a third pass on the back side, and a fourth and final pass on the remaining unsprayed left or right side. A person first faces the plurality of spray nozzles 14 and initiates a spray sequence by pushing a button 10 on the front of the panel 15. When the actuating button 10 is depressed, the spraying cycle is initiated in the apparatus 2. FIG. 3 is a rear perspective view of the panel 15. A control device 12 containing programmable algorithms that control the operation of the plurality of spray nozzles 14 starts to execute its programmed spraying sequence, and skin surfaces 11 facing the nozzles 14 are sprayed with the atomized mixture 5 on a first pass. After this sequence has finished, a delay period provides time for the person P to turn 90 degrees and another side toward the plurality of nozzles 14 before the control device 12 initiates the second pass spraying sequence. It is preferred that the programmed delay period is about three to twelve seconds between spray sequences. However, any delay time period may be chosen as is appropriate for use with the present invention. During each delay period if a person P turns 90° to present an unsprayed side to the nozzles 14, at the end of the four pass sequence the person should be thoroughly sprayed with the atomized mixture 5.

It will be apparent to one skilled in the art that other arrangements of the person P in front of the nozzles may be accomplished by the present invention. For example, in some applications, only two passes may be required to adequately spray a person; accordingly, the control unit 12 may be programmed to provide only two discreet spray sequences separated by only one delay period, the person P turning around 180° and presenting opposite sides to the nozzles 14 during each spray sequence. Or the control panel may only initiate one spray sequence for each signal by the button 10.

With reference again to FIGS. 1 and 2, according to the present invention, the duration and amount of liquid product sprayed through the plurality of nozzles 14 is determined by the desired amount of product to be received by the person P. Other factors would also include the specific viscosity of the fluid, or the ratio of air to fluid mixture required for a desired application pattern.

It is preferred that the matrix 18 is comprised of nine discrete rows 20₁ through 20ₙ, where n is the number of rows 20. In the present embodiment, four nozzles 14 are in each row 20. In a typical application, the control module 12 or controlling means 12 fires each row 20 individually, one after another. A preferred pattern starts with a bottom row 20ₙ and proceeds to the top row 20₁ sequentially. In other words, the bottom row of jets 20ₙ fires and, when it has completed firing, then the next higher row of jets 20ₙ₋₁ immediately starts firing. As described earlier, the length and duration of each spray routine is controlled by the panel 12, and panel 12 is programmed according to the amount of mixture required to be deposited upon the person P for each specific mixture. Therefore, the duration of each row of jets 20 is typically the same, and this duration period is programmed according to the fluid and the amount of coverage desired for the specific fluid by the atomized spray mixture. It will be readily apparent that other arrangements can be programmed into the control module 12, wherein some rows may have a shorter duration spray epoch than other rows. For example, if the sequence were to start with the top row 20₁ of nozzles firing and then sequentially moving downward since an atomized mixture may remain in the air, it may be found that shorter spray bursts may be required by the lower nozzles. One skilled in the art may readily determine the optimum spray period or epoch for each row of nozzles 20 and the present invention is not limited to one where all the rows 20 are sprayed for one common fixed epoch.

Similarly, other patterns may be programmed into the control module 12. For example, an every other pattern may be programmed wherein odd row nozzles are fired from top to bottom, or from bottom to top, or from middle to top to bottom in a non-up and down sequential pattern and then even rows of nozzles fired thereafter. The exact row and sequence of nozzles is not important according to the present invention. What is important is that a multiplicity of nozzles is utilized in a user programmed pattern to provide a desired dispersal of atomized fluid mist. Also, where a person of short stature uses the invention, the top row 20₁ and second row 20₂ may not be required to effect a complete spray sequence. Accordingly, a selection option may be provided to select the number of rows 20 used by each spray sequence. In one embodiment, a three-position switch or a user programmable controller 13 allows a user to select between eight, nine and ten row 20 spray sequence passes: one switch 13 setting disables rows 20₁ and 20₂ to provide an eight-row sequence; a second switch 13 setting disables row 20₁ only to provide a nine-row sequence; and the last setting enables all ten rows 20.

In some embodiments of the invention, the nozzle rows 20 may be broken down into discreet groupings within the row, or the nozzles may fire independently. For example, row 20₄ may comprise a middle row 24 and outer nozzle set 22, wherein row 24 and nozzle set 22 may be fired by the controller 12 differently. Where the person P has a thin profile, the controller 12 may responsively fire only the middle row 24 and not fire the outer nozzle set 22 at all; or, alternatively, the duration or amount of fluid expelled by the outer group 22 may be decreased. This will allow an efficiency of fluid application wherein due to the narrow profile of the person P, the dispersal pattern does not require that each row uniformly expel the same amount of atomized fluid fire across the entire width of a row 20.

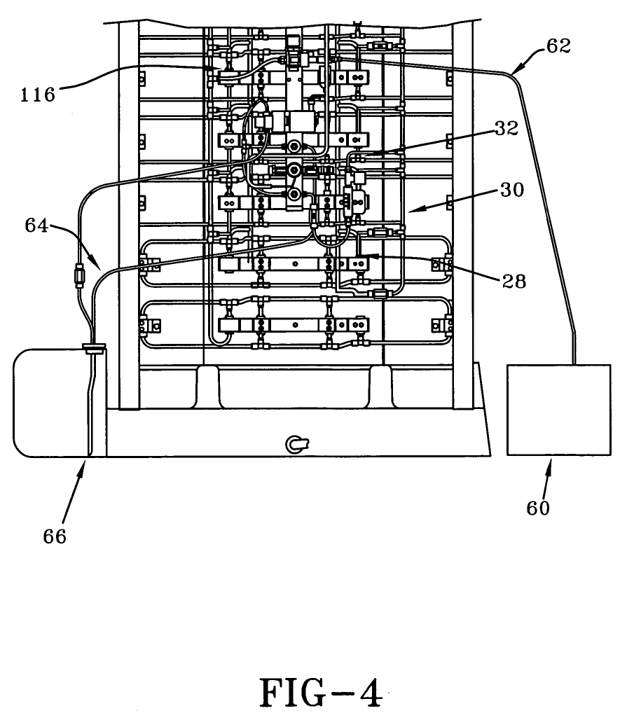

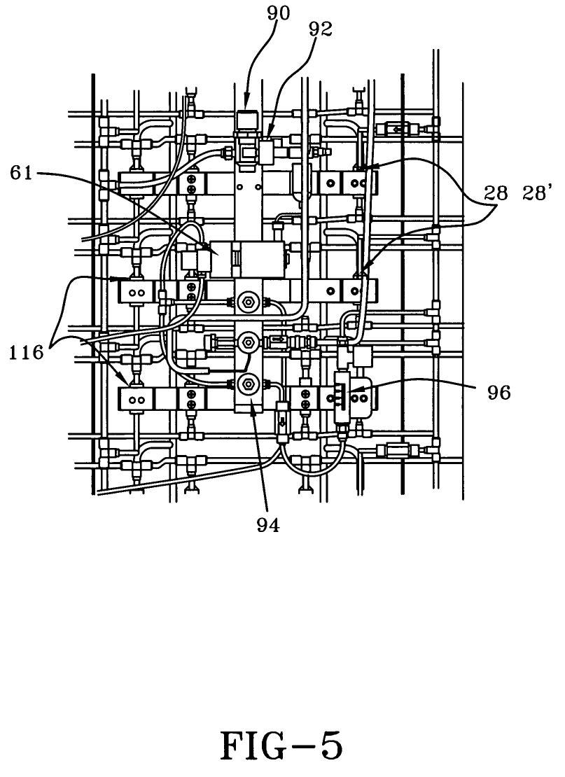

With reference to FIGS. 1 through 5, in the present embodiment of the invention, each row of nozzles 20 comprises four nozzles 14. It will be readily apparent that other numbers may be available for each row 20, and that the rows 20 may all have the same or different numbers of nozzles 14. Where a row of nozzles is commonly fired, it is preferable to plumb them all through the same piping apparatus. FIGS. 4 and 5 provide detail views of the back on panel 15. In the present embodiment, each row 20 of nozzles 14 is commonly plumbed by one pipe 30 or conduit 30. An advantage of operating the nozzles 14 in discreet rows 20 is that the amount of air pressure required by the present invention is kept to a low amount. Where more than one row 20 is fired by an available air driving mechanism, then more air pressure would be required. This would require a larger air compressor or larger reservoir of compressed air in order to use the present invention 2. But by firing each row 20 singly, one at a time, the present embodiment is efficient in its use of air. This insures that all of the nozzles in any given row 20 will fire at the same time and that each will receive the same fluid in the same amounts driven by the same air pressure. In alternative embodiments of the present invention wherein a row 20 may be subdivided into a fractional row 22 or group 24, the nozzles in a row 22 or a group 24 may be commonly plumbed. It is to be understood that a “common plumb” configuration is not required to practice the present invention. It is just an efficient and economic embodiment of the present invention.

In a preferred sequence, as the first row of nozzles 20 turns on, the compressed air is initiated and compelled through the nozzles 14 for about one second. After this one second period, the fluid to be atomized is then piped into the nozzles by separate pipe 32, also for another second of duration. Lastly, after the fluid is turned off to the nozzles 14 in the row being initiated 20, then air continues to be compelled through the nozzles 14 for about one more additional second. This insures that the fluid is not projected out of the nozzle without air wherein a solid stream of fluid would be compelled, much like a squirt gun. This also ensures that the nozzle is cleaned out after every spray.

In the present embodiment, it is preferred that each row 20 of nozzles 14 compels an atomized mixture at a total of about 15 cubic feet per minute (cfm). Where each row 20 has four nozzles 14, each nozzle 14 then compels one-fourth of 15 cfm, or about 3.75 cfm. Alternatively, the present embodiment can be configured to simultaneously fire all of the nozzles 14 located on the panel 15. Where forty nozzles 14 are present, to operate each at 3.75 cfm would require 150 cfm of air flow. Thus, the operator of the apparatus 2 can select the rate at which the firing sequence can be completed based upon the availability of pressurized air. To be efficient in terms of air pressure requirements, the one-row-at-a-time sequence can be selected. However, where time is of the essence and sufficient air pressure is available, the simultaneous operation, or multiple row operation options can be selected. It is to be understood that other nozzle sizes may be practiced under the current invention. For example, each nozzle 14 may be able to accommodate less than 3-4 cfm or significantly more than 3-4 cfm.

It has been found that a preferred pattern of row selection by the control module 12 is to have the bottom row 20 of nozzles fire first and then each row above fire sequentially from bottom to top. This gives the user P notice that the jets are firing and allows the person to become accustomed to the sound and pressure of the atomized mixture as it strikes his body P well before the mixture reaches the face area. This is desirable in that the person is not surprised by a sudden blast of liquid and air mixture about the face and eyes prior to the person closing his eyes before being struck about the face. This also lets the person calmly receive the area around the mouth and nose area with full notice of the rate of the cascading pattern as it rises up upon the persons' body. Thus, the present invention's pattern of bottom to top spraying allows a user P to receive the mixture when ready about the upper areas and not be startled by a sudden blast or noise from the multiple jets 14.

With continued reference to all of the FIGS. and especially to FIG. 4, in the present embodiment, one reservoir 60 contains the liquid solution to be conveyed through the multiple nozzles 14. A second air supply system is also connected through piping 62 into the array of spray nozzles 14. Where the liquid solution is a tanning solution, a typical application results in about 100-200 milliliters of fluid material total being conveyed through all of the jets 14 during one complete application of two cascades of atomized material upon a person P, that is one cascade for the front area, and one cascade pattern for the back area or four cascades for a four pass system.

The amount of fluid and air compelled through each nozzle, and the pressure at which the air is driven through the nozzle 14, is controlled by the user through controls on the back of the panel 15. Air pressure regulators 90 with pressure gauges 92 enable a user to easily adjust the regulator and have the air pressure gauge 92 indicate a desired air pressure for expelling air through the nozzle 14. Similarly, there is a needle valve 94 wherein a flowmeter 96 may be set to a desired quantity of fluid that will be expelled by each nozzle at the specified rate as indicated by the flowmeter 96. The duration of the air pressure application for each row 20 of nozzles 14 is controlled by a timing knob that is located on the back panel. Thus, a user may easily adjust the amount of fluid, the air pressure, and the duration of the air pressure and fluid application by the present invention 2. These settings may be adjusted according to the type of fluid conveyed from the reservoir 60 and, therefore, responsively adjusted to the viscosity or desired application amount required by each type of fluid. Although in the present embodiment all of the nozzles 14 are operated for the same time period; thus, only one time controller setting is required. In alternative embodiments, more than one time control unit may be attached to the invention 2. For example, each row may have its own individual time controller unit. Similarly, each row of nozzles 20 may have its own fluid flowmeter 96 and air pressure gauge 92, or each nozzle 14 may have its own set of fluid needle valves and air pressure regulators 94 and 90.

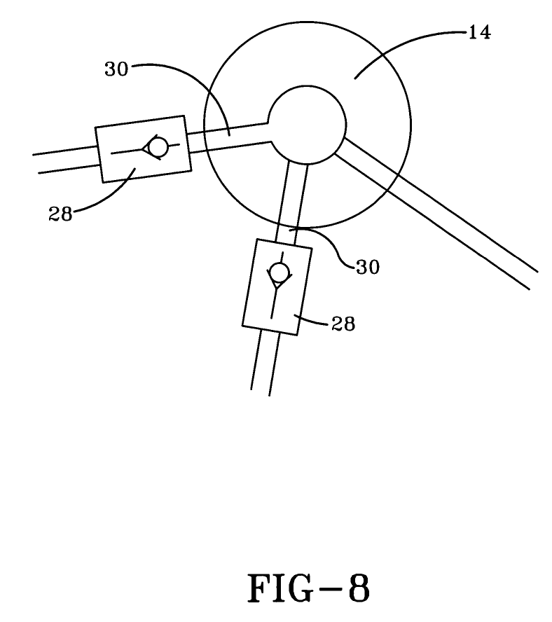

With reference to FIG. 3 through 5 and also to FIG. 8, the nozzles 14 may be accompanied by shut off valves 28 that are disposed between the piping or conduit 30 and the nozzles 14. That is to say that the shut off valves 28 may be connected in line with the conduit 30 thereby controlling fluid flow to the nozzles 14. Each nozzle 14 may have its own single shut off valve 28. However, it is contemplated that a complete row or bank of nozzles 14 may have a shut off valve 28. In one embodiment, the shut off valve 28 may be a check valve 28′ that automatically allows flow in one direction and prevents flow in the reverse direction. It can be seen that the check valve 28′ prevents air and fluid flow back into the conduit 30 from which the respective fluid is flowing. This is especially useful for use with multiple conduits 30 connected to a single nozzle 14 as described in detail in the following paragraph. The shut off valve 28 or check valve 28′ may be useful for purging the nozzle 14. By purging it is meant that a stream of pressurized air, or other cleansing fluid, may be directed through the nozzle 14 to remove the residual tanning fluid within the nozzle 14 and adjacent conduit 30. It is noted for clarity that fluid from the reservoir 66 is prevented from being communicated to the nozzle during the purging process. This is especially useful for use when multiple different fluids are used in conjunction with a single nozzle 14.

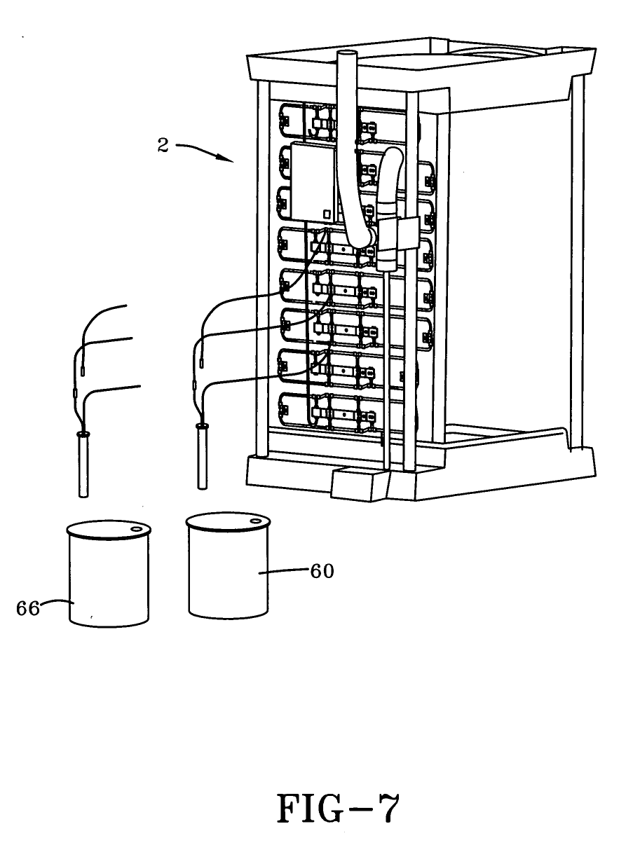

With continued reference to FIG. 3 through 5 and now to FIG. 7, a single nozzle 14 may have communicated to the nozzle 14 one or more conduits 30 for use in channeling one or more fluids, to be atomized by pressurized air, from multiple reservoirs 60, 66. In one embodiment, two reservoirs may be incorporated. In this manner, first and second reservoirs 60, 66 may contain the same fluid or may each contain a different fluid. One reservoir 60 may contain a first associated fluid and the second reservoir 66 may contain a second different associated fluid. For example, the plurality of fluids may differ by tanning darkness. That is to say, that one fluid, when applied in the manner described herein, may provide a darker shade of tanning than the second fluid. It is noted that the controlling means 12 may be programmed to selectively and/or remotely select which fluid is to be applied without having to manually change the reservoir each time the spraying device is utilized. Alternately, if the same fluid is contained within each reservoir 60, operation of the device may not be halted when the reservoir is empty, as is the case when the single reservoir device is used. In this manner, the controlling means 12 may detect that the first reservoir 60 is empty and may automatically switch to the second reservoir 66 allowing time for the operator to replace the empty reservoir. Clearly, it can be seen that any number of fluid reservoirs may be chosen with sound judgment as is appropriate for use with the subject invention. It is contemplated in an alternate that one reservoir 60 may be connected to a first group of nozzles 14 or rows or nozzles, and that a second reservoir 66 may be connected to a second group of nozzles 14 for use in selectively applying different atomized fluids.

With reference to FIGS. 2 and 3, the central control mechanism 12 or spray nozzle controlling means 12 contains the controller systems, microprocessors for controlling the various elements, and power supply elements. It fits into a bracket that also is designed to mount into the same bracket receiving holes 110 as are used by the nozzles 14 and the bracket 112 that is used to hold the air pressure regulators 90, and fluid needle valves 94.

It should be noted that, although the present invention embodiment 2 is configured to spray tanning solution upon a user P, any type of fluid and air mixture may be deposited upon a user P with the present invention 2. For example, water can be used to cool off a user P on a hot day, or one who has been exerting and is overheated due to physical activities. Or, bug repellant or other types of solutions may be deposited upon a person P. Also, sun block may be deposited upon a user P at a beach or sunny area. Or one exposed to hazardous materials may have an antidote or neutralizing solution deposited upon the person, thereby detoxifying the person. In this way, the invention can be used by materials at hazardous responders' incidents to decontaminate somebody exposed to dangerous chemicals or solutions.

One master air regulator 90 is used to control the air delivered to the multiple nozzles 14. Each of the nozzles 14 has an air solenoid structure 116. The pumping mechanism 61 has a bypass system 64 wherein excess pressure is returned back into the pump reservoir 66. This is desired in order to keep excess pressure from being applied to the fluid, in excess of that required to deliver the fluid to the nozzles 14. Otherwise, this system serves an additional function in that the bypass system 64 returns fluids to the reservoir 66, and the pressure with which the bypassed fluid enters the reservoir 66 contributes to keeping the fluids mixed within the reservoir 66 by providing a mixing action. Thus, the bypass system 64 constantly recirculates the fluid in the reservoir 66.

Alternatively, the controller 12 can be used in conjunction with an input function wherein the size and stature of the user P is inputted into the system 12 to select the number of nozzles fired by the controller 12. For example, an electric eye or visual scan system (not shown) may process an image of the person standing in the booth enclosure 4. Where that person is short, the top rows 20₁ or 20₂ may not fire at all. Or a four jet row 20₁ may be instructed to fire only two nozzles in the center of the row. Similarly a very thin person may require only two center nozzles in any given row based upon an input to the system 12.

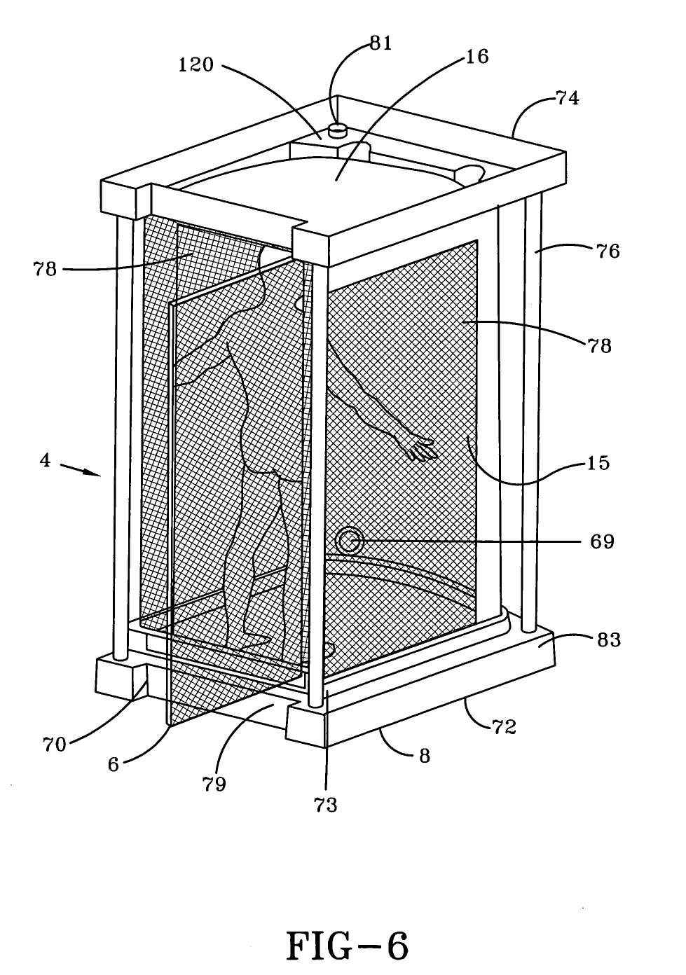

It may be desirable to practice the present invention within an enclosed structure, such the booth 4 illustrated in FIG. 6. An enclosure may provide a means of privacy for a user P. It may also provide a means to control ambient air currents, thus preventing side winds or currents from interfering with the atomized mixture 5 within the engagement region 3. And where containment of the atomized mixture 5 is desired, it may function to contain the mixture 5 within the booth, thus preventing undesirable spreading of the atomized mixture 5 beyond the engagement region 3. The excess atomized mixture 5 may be allowed to vent through passive vents 68, or it may be collected through a collection means 69. One collection means 69 is an impeller vacuum pump that removes the mist 5 from the booth 4 and expels it to a collection means (not shown) or into the outside atmosphere.

With reference now again to FIG. 6, the booth enclosure 4 is configured for receiving a person who will stand vertically within the booth 4. The booth 4 and nozzles 14 are configured to provide the engagement region 3 at the rear of the booth 4, leaving the front area of the booth for the volatile mixing region 7. Non-skid surfaces 8 are applied to the base 72 floor surface, in order to prevent persons utilizing the apparatus 2 from slipping and falling on precipitated mixture 5 accumulating on the floor 72. The person P then closes the door 6 and reaches in front of his position and presses actuating button 10. A showerhead 16 may be provided at the top of the booth 4 to convey water to wash down the interior of the booth 4 after completion of the spraying operations, thereby washing condensed atomized mixture from the internal surfaces of the spray booth 14 enclosure. Alternatively, a mixture of water and another fluid, such as bleach or soap, may also be conveyed through the showerhead 16. Or, also, a disinfectant or disinfectant in water solution may be conveyed through the showerhead 16. In this way, the present invention enables an operator or supervisor to trigger a cleaning cycle that will clean unused atomized fluid from the inside of the machine and also disinfectant and sanitize the interior walls and floor mat 8 for a subsequent user of the booth. A floor drain 73 may be installed in the base 72 of the booth 4 to collect rinsed or precipitated solution.

Another feature of the present invention is a trough 70 that is formed in the base 72 of the spray booth 4 at the door opening at the base of the door 6. This enables fluids running down the door 6 to be accumulated in the trough 70 and then redirected back towards the base of the spray booth 72. This keeps the doorway region clear of fluid and spills of fluids, thus improving the safety of the present invention by reducing the risks of slips and falls from fluids outside of the booth by the door area.

In a preferred embodiment, the booth 4 has a simple and efficient design. The bottom tray 72 or bottom booth portion 72 and top tray 74 or top booth portion 74 of the present embodiment 2 are formed by the same mold or process out of a plastic material. In this case, 72 and 74 can be interchanged during construction and during manufacturing process, allowing for efficiencies and economies of scale. The bottom and top trays 72 and 74 are connected to each other by four extruded aluminum poles 76. In between the top and bottom trays 72 and 74 are three bent translucent plastic panels 78. The four poles 76 provide the rigid structure to the assembly 2.

With reference again to FIGS. 2 and 6, in the present invention, there is a vent 68 located in the back panel 78 wherein the pressure expelled by the nozzles 14 and excess atomized mixture 5 may be freely vented outward into the outer atmosphere surrounding the booth 4. No means are required to filter or otherwise collect the atomized mixture 5 being vented, although such a means or apparatus may be added. The atomized spray mixture 5 that does not land on and become retained by person P, or become vented out through the vent 68, will settle and condense upon the glass panels 78 and bottom surface of the tray 72, and then slowly flow towards the floor drain 73. Similarly, a negative pressure system powered fan system 69 may be used to apply a vacuum to the booth and suck out or remove the excess atomized mixture 5. It will also be readily apparent that this excess fluid may then be collected into some type of collection system (not shown) wherein the excess fluid can be disposed of, recycled, or otherwise contained and removed from the atmosphere. It is may also be preferred that the entire panel 15 may be removed and replaced with a different panel. Thus, the booth 4 can be quickly and easily altered to provide a different array of nozzles 14 and control mechanisms 12, and regulators 90 and 92, etc.

In a preferred embodiment, the door 6 and side panels 78 are all formulated from polycarbonate and acrylic materials, although it is readily apparent that glass or other rigid translucent materials may be used. By using translucent materials for the door 6 and side panels 78, light can be provided into the enclosure 4 from outside sources, either through general room lighting or through sunlight. Alternatively, mirrored or one-way opaque panels 78 and door 6 may be used wherein light will be admitted into the enclosure 4; however, a user will not be readily apparent to a viewer from outside the enclosure.

Alternatively, a lighting system 120 may be inserted into the enclosure 4. In the present embodiment, a 12-Volt lighting system 120 is utilized. It is preferred that all of the power systems are operated on a common 12-Volt system; however, other voltages may be practiced under the current invention. Where internal lighting systems are present, the walls 78 and door 6 may be opaque, thus affording a user improved privacy while using the invention apparatus 2.

Suitable materials for the top and bottom trays 72 and 74 are hand-laid fiberglass composite materials, with a foot per unit measurement well known to those skilled in the art. The extruded aluminum poles 76 are typically one-eighth inch thick hollow cylindrical tubes, although other materials of other thicknesses may also be used under the present invention. The clear panel 78 is joined with aluminum brackets 79, typically one-quarter inch in thickness, although once again other materials may be used to join the panels 78 and door 6 together. In the present embodiment, rapid assembly of the structure 2 can be achieved by putting threaded studs 81 in the top and bottom trays 72 and 74. The side panel 78 and door panel assembly 6 slide into slots 83 within the trays 72 and 74 and the aluminum poles are threaded onto the studs 81, thereby clamping the trays 74 and 72 together and thereby clamping the entire structure together.

While preferred embodiments of the invention have been described herein, variations in the design may be made, and such variations may be apparent to those skilled in the art of making tools, as well as to those skilled in other arts. The materials identified above are by no means the only materials suitable for the manufacture of the tool, and substitute materials will be readily apparent to one skilled in the art. The scope of the invention, therefore, is only to be limited by the following claims.

What is claimed is:

1. A spraying device, comprising:
an upright structure having a nozzle support portion;
at least first and second spray nozzles being operatively attached to the nozzle support portion;
means for operatively supplying an associated fluid to the at least first and second spray nozzles;
a spray nozzle controlling means for use in selectively independently actuating the at least first and second spray nozzles respectively; and,
wherein when the spraying device is engaged the at least first spray nozzle is sequentially actuated with respect to the at least second spray nozzle.

2. The device of claim 1, wherein the upright structure is an enclosure for use in containing the associated fluid, and,
wherein the nozzle support portion is a wall member.

3. The device of claim 2, wherein the enclosure is a modular booth having:
a plurality of spray containing walls;
a bottom booth portion; and,
a top booth portion.

4. The device of claim 2, wherein the at least first and second spray nozzles are at least first and second banks of spray nozzles.

5. The device of claim 4, wherein the at least first bank of spray nozzles are fixedly operatively attached with respect to the wall member.

6. The device of claim 4, wherein the means for operatively supplying an associated fluid comprises:
pumping means for use in conveying the associated fluid to the at least first and second banks of spray nozzles; and,
at least a first reservoir operatively connected to the pumping means.

7. The device of claim 6, further comprising:
a shut-off valve operatively communicated between the at least a first reservoir and the at least first spray nozzle.

8. The device of claim 7, wherein the shut-off valve is a check valve.

9. The device of claim 6, further comprising:
conduit for use in channeling the associated fluid, the conduit being operatively connected to the pumping means and the at least first and second spray nozzles;
wherein the pumping means is a mechanical pump that continuously cycles the associated fluid; and,
further comprising:
a bypass valve for use in selectively channeling the associated fluid, the bypass valve being operatively communicated between the pump and the at least first and second spray nozzles.

10. The device of claim 6, further comprising:
at least a second reservoir operatively connected to the pumping means.

11. A method for dispensing an associated fluid, comprising the steps of:
providing at least a first and second spray nozzles, wherein the at least a first spray nozzle is selectively independently actuate-able with respect to the at least a second spray nozzle;
providing means for use in pumping an associated fluid from at least a first reservoir, the pumping means being operatively communicated to the at least first and second spray nozzles;
providing controlling means operatively communicated to actuate the at least first and second spray nozzles;
engaging the controlling means;
spraying an associated fluid through the at least a first spray nozzle; and,
sequentially spraying the associated fluid through the at least a second nozzle.

12. The method of claim 11, wherein before the step of spraying an associated fluid through the at least a first spray nozzle, further comprising the step of:
atomizing the associated fluid.

13. A method for dispensing fluid, comprising the steps of:
providing at least a first spray nozzle;
providing pumping means for use in pumping associated fluid, the pumping means being operatively communicated to the at least a first spray nozzles;
providing controlling means operatively communicated to actuate the at least a first spray nozzles;
providing first and second fluid reservoirs operatively communicated to the pumping means;
selecting one of the first and second reservoirs from which to draw associated fluid from;
engaging the controlling means; and,
spraying the associated fluid through the at least a first spray nozzle.

14. The method of claim 13, further comprising the steps of:
purging the associated fluid from the at least a first spray nozzle; and,
spraying associated fluid from the remaining of the one of the first and second reservoirs.
